(19) United States
(12) Patent Application Publication
(10) Pub. No.: US 2023/0051903 A1
(43) Pub. Date: Feb. 16, 2023

(54) FERMENTATION METHOD

(71) Applicant: CARGILL, INCORPORATED, Wayzata, MN (US)

(72) Inventors: Christian BARTELS, Council Buffs, IA (US); Matthew GUTZMANN, Blair, NE (US); Christopher Lee NEUMANN, Omaha, NE (US); Eric PAQUETTE, Dayton, OH (US); Victor SHERONY, Omaha, NE (US); Joseph SPENCER, Bennington, NE (US)

(73) Assignee: CARGILL, INCORPORATED, Wayzata, MN (US)

(21) Appl. No.: 17/758,908

(22) PCT Filed: Jan. 15, 2021
(86) PCT No.: PCT/US2021/013559

§ 371 Date: Jul. 15, 2022

Related U.S. Application Data
(60) Provisional application No. 62/962,563, filed on Jan. 17, 2020.

(51) Int. Cl. C12P 7/14; C12P 7/40; C12P 19/00

(52) U.S. Cl. CPC C12P 7/14; C12P 7/40; C12P 19/00

(57) ABSTRACT

A fermentation process includes contacting a starch hydrolysate with a glucoamylase with agitation. And allowing for settling to form a multi-phase solution. A first phase of the multi-phase solution includes a saccharide component comprising about 30 wt % to about 80 wt % (e.g., 30 wt % to 70 wt %, 30 wt % to 60 wt %) based on a total carbohydrate present. The method further includes draining the first phase to isolate the first phase from a second phase to form a fermentation broth comprising a first portion of the first phase. The method further includes fermenting the fermentation broth until a concentration of glucose in the fermentation broth is 40 g/L or less. The method includes adding a second portion of the first phase to the fermentation broth to maintain a concentration of glucose in a range of from about 1 g/L to about 20 g/L in the fermentation broth.

### BACKGROUND

Fermentation processes are used commercially at large scale to produce organic molecules such as ethanol, citric acid and lactic acid. In those processes, a carbohydrate is fed to a microorganism that is capable of metabolizing it to the desired fermentation product. The carbohydrate and microorganism are selected together so that the microorganism is capable of efficiently digesting the carbohydrate to form the product that is desired in good yield. It is becoming more common to use genetically engineered microorganism in these processes, in order to optimize yields and process variables, or to enable particular carbohydrates to be metabolized.

### SUMMARY OF THE INVENTION

Various examples of the present disclosure provide a fermentation process. The process includes contacting a starch hydrolysate with a glucoamylase with agitation. The process further includes ceasing the agitation to allow for settling to form a multi-phase solution. A first phase of the multi-phase solution includes a saccharide component and has about 30 wt % to about 60 wt % glucose based on total carbohydrate present in the first phase and a second phase of the multi-phase solution includes a higher amount of total suspended solids than the first phase. The method further includes draining the first phase following formation to isolate the first phase from the second phase. The method further includes heating at least the first phase to a temperature of at least 70° C. A fermentation broth is formed and includes a first portion of the first phase. Initially, a concentration of glucose in the fermentation broth is typically in a range of from about 55 g/L to about 95 g/L (for example in a fed-batch fermentation it can be in a range of from about 55 g/L to about 95 g/L, or about 60 g/L to about 70 g/L and in a batch fermentation it can be in a range of from about 60 g/L to about 95 g/L or from about 70 g/L to about 90 g/L). The method further includes fermenting the fermentation broth with a microorganism to produce a fermentation product until the concentration of glucose is 40 g/L or less. Thereafter, the method further includes contacting the fermentation broth with glucoamylase. Thereafter, the method further includes adding a second portion of the first phase to the fermentation broth to maintain a concentration of glucose in a range of from about 1 g/L to about 20 g/L in the fermentation broth. In some preferred examples the fermentation product is substantially free of ethanol.

Various examples of the present disclosure provide a fermentation process. The process includes contacting a starch hydrolysate with an alpha-amylase before contacting the starch hydrolysate with glucoamylase. The starch hydrolysate has a dextrose equivalent number in a range of from about 8 to about 13 prior to adding glucoamylase. The method further includes contacting the starch hydrolysate with a glucoamylase with agitation. The method further includes ceasing agitation for a sufficient amount of time to allow for settling to form a multi-phase solution. A first phase of the multi-phase solution includes a saccharide component having about 30 wt % to about 60 wt % glucose based on total carbohydrate present in the first phase and a second phase of the multi-phase solution comprising a higher total suspended solids amount than the first phase. Settling is performed for a time in a range of from about 5 hours to about 13 hours. The method further includes draining the first phase to isolate the first phase from the second phase. The method further includes heating at least the first phase to a temperature of at least 70° C. A fermentation broth is formed and includes a first portion of the first phase. Initially, a concentration of glucose in the fermentation broth is typically in a range of from about 55 g/L to about 95 g/L, or about 60 g/L to about 70 g/L and in a batch fermentation it can be in a range of from about 60 g/L to about 95 g/L or from about 70 g/L to about 90 g/L. The method further includes fermenting the fermentation broth with a strain of yeast with deletions or disruptions for one or more pyruvate decarboxylase genes such that the pyruvate decarboxylase activity in the strain is substantially eliminated to produce substantially no ethanol. The yeast, however can produce at least 50 g/L, 60 g/L, 70 g/L, 80 g/L, or 90 g/L of a fermentation product comprising lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof until a concentration of glucose in the fermentation broth is 40 g/L or less. The fermentation product preferably comprises lactic acid and salts thereof, with the lactic acid and salts thereof. Thereafter the method further includes contacting the first phase with glucoamylase in the fermentation broth and adding a second portion of the first phase to the fermentation broth to maintain a concentration of glucose in a range of from about 1 g/L to about 20 g/L. During this period when the glucose is in a range from 1 g/L to about 20 g/L, and as the fermentation proceeds and if the fermentation product is an organic acid, the pH typically is maintained from 4 to 5 (for example from 4.2 to 4.6, from 4.3 to 4.5), and preferably at a pH of about 4.4 by the addition of calcium hydroxide (lime). Once a sufficient amount of lime has been added the fermentation pH is allowed to free fall to the desired final pH. The amount of lime added is determined by the expected fermentation product concentration (e.g. lactic acid and salts thereof) at the end of fermentation and the level of free acid desired. In some preferred examples the fermentation product is substantially free of ethanol.

Various examples of the present disclosure provide a fermentation process. The process includes contacting a starch hydrolysate with a glucoamylase with agitation. The process further includes ceasing the agitation to allow for settling to form a multi-phase solution. A first phase of the multi-phase solution includes a saccharide component and has about 30 wt % to about 60 wt % glucose based on total carbohydrate present in the first phase and a second phase of the multi-phase solution includes a higher amount of total suspended solids than the first phase. The method further includes draining the first phase following formation to isolate the first phase from the second phase. The method further includes heating at least the first phase to a temperature of at least 70° C. A fermentation broth is formed and includes a first portion of the first phase. Initially, a concentration of glucose in the fermentation broth is typically in a range of from about 55 g/L to about 95 g/L, or about 60 g/L to about 70 g/L and in a batch fermentation it can be in a range of from about 60 g/L to about 95 g/L or from about 70 g/L to about 90 g/L. The method further includes fermenting the fermentation broth with a microorganism until a concentration of glucose in the fermentation broth is 40 g/L or less. Thereafter, the method further includes contacting the fermentation broth with glucoamylase. In some preferred examples the fermentation product is substantially free of ethanol.

Various examples of the present disclosure provide a fermentation process. The process includes contacting a starch hydrolysate with an alpha-amylase before contacting the starch hydrolysate with glucoamylase. The starch hydrolysate has a dextrose equivalent number in a range of from about 8 to about 13 prior to adding glucoamylase. The method further includes contacting the starch hydrolysate with a glucoamylase with agitation. The method further includes ceasing agitation for a sufficient amount of time to allow for settling to form a multi-phase solution. A first phase of the multi-phase solution includes a saccharide component has about 30 wt % to about 60 wt % glucose based on total carbohydrate present in the first phase and a second phase of the multi-phase solution comprising a higher total suspended solids amount than the first phase. Settling is performed for a time in a range of from about 5 hours to about 13 hours. The method further includes draining the first phase to isolate the first phase from the second phase. The method further includes heating at least the first phase to a temperature of at least 70° C. A fermentation broth is formed and includes a first portion of the first phase. Initially, a concentration of glucose in the fermentation broth is typically in a range of from about 55 g/L to about 95 g/L, or about 60 g/L to about 70 g/L and in a batch fermentation it can be in a range of from about 60 g/L to about 95 g/L or from about 70 g/L to about 90 g/L. The method further includes fermenting the fermentation broth with a strain of yeast with deletions or disruptions for one or more pyruvate decarboxylase genes such that the pyruvate decarboxylase activity in the strain is substantially eliminated to produce substantially no ethanol. The yeast, however can produce at least 40 g/L, 50g/L, 60 g/L, 70 g/L, 80 g/L, or 90 g/L of a fermentation product comprising lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof until a concentration of glucose in the fermentation broth is 40 g/L or less. Thereafter, the method further includes contacting the fermentation broth with glucoamylase. In some preferred examples the fermentation product is substantially free of ethanol.

### DETAILED DESCRIPTION OF THE INVENTION

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

The term “about” as used herein can allow for a degree of variability in a value or range, for example, within 10%, within 5%, or within 1% relative to a stated value or of a stated limit of a range, and includes the exact stated value or range.

The term “substantially” as used herein refers to a majority of, or mostly, as in at least about 90%, 95%, 96%, 97%, 98%, 99%, 99.5%, 99.9%, 99.99%, or at least about 99.999% or more, or 100%.

According to various examples of the present disclosure, a fermentation process includes subjecting a starch hydrolysate composition to a first partial saccharification process to yield two phases or multi-phase solution. A first phase of the two phases includes a sufficient amount of glucose to commence fermentation and a sufficient amount of saccharides (e.g., oligomers or polymer of glucose) so that it can be later used in a second fermentation process such as a simultaneous saccharification and fermentation (SSF) process. The fermentation process or method as a whole is typically classified as a batch or batch-fed process.

Starch is a widely available and inexpensive saccharide/carbohydrate source. It is available from a wide variety of plant sources such as corn, wheat, rice, barley, potatoes, cassava, and the like. Many organisms (e.g., microorganisms) are not capable of metabolizing starch directly, or else metabolize it slowly and inefficiently. Accordingly, it is can be helpful to treat starch before feeding it into a fermentation process, in order to at least partially break it down into a suitable amount of glucose that the microorganism can ferment easily.

As an example, starch is hydrolyzed to any suitable degree to form a mixture including a saccharide component that includes mainly glucose (e.g., dextrose). However, complete hydrolysis to glucose can be difficult and it is possible for the mixture to include other saccharides such as fructose, maltose, isomaltose, panose, and other DP3 and DP4 (e.g., oligomers of glucose). In various examples, the major type of monosaccharide present is glucose.

In the present fermentation process, a starch hydrolysate is processed to at least partially be liquified and subjected to partial saccharification. This operation in the fermentation process can include contacting the starch hydrolysate with an amylase, which is an enzyme that catalyzes the hydrolysis of starch. An example of a suitable amylase for this operation is alpha-amylase (classified under EC 3.2.1.1). The alpha amylase acts to hydrolyze at least some saccharides from the starch and begin to make the starch more accessible for further processing. This can be further aided by heating the starch hydrolysate (e.g., to denature proteins present in the starch hydrolysate and unfold the starch hydrolysate). For example, the starch hydrolysate is typically heated to a temperature of at least about 80° C., at least about 100° C., in a range of from about 80° C. to about 130° C., less than about 80° C., 85, 90, 95, 100, 105, 110, 115, 120, 125, or about 130° C., or greater than about 80° C., 85, 90, 95, 100, 105, 110, 115, 120, 125, or about 130° C.

After exposure to alpha-amylase, a dextrose equivalent number of a solution including the starch hydrolysate is typically in a range of from about 1 to about 15, about 8 to about 13, less than about 5, 6, 7, 8, 9, 10, 11, 12, 13, 14, or about 15 or greater than about 5, 6, 7, 8, 9, 10, 11, 12, 13, 14, or about 15. As understood herein, dextrose equivalent is a measure of the amount of reducing sugars present in a sugar product, expressed as a percentage on a dry basis relative to dextrose.

The starch hydrolysate is subjected to further partial saccharification by contacting the starch hydrolysate with a glucoamylase. A glucoamylase is an exoamylase, which cleaves 1,4-α-glycosidic bonds from the nonreducing end of the glycosidic chains releasing d-glucose, thus increasing the content of fermentable sugars and reducing the nonfermentable dextrins in the hydrolysate. Contacting the starch hydrolysate with glucoamylase is accompanied by agitating the starch hydrolysate (e.g., by mixing or otherwise shearing the hydrolysate). The saccharification of the starch hydrolysate is carried out in such a manner that a multi-phase solution (e.g., a two-phase solution) is formed. A first phase includes glucose and other saccharides that are hydrolyzed from the starch. A second phase includes wastes from the starch hydrolysate such as proteins, lipids, and the like. In a container, the second phase is located towards the top of the container and the first phase is located towards the bottom of the container.

Forming the multi-phase solution requires a carful balance of agitating the starch hydrolysate and then controlling the amount of time that the agitated solution is allowed to settle to ultimately form the multi-phase solution. Moreover, controlling the time of agitation and settling can also help to control the weight percent of glucose present based on total carbohydrate present in the first phase. The time that agitation and settling are each allowed to occur over can depend on several factors including the volume of the container in which settling and agitation are carried out in. As an example, agitation can occur over a time in a range of from about 3 hours to about 10 hours, about 5 hours to about 8 hours, less than about 4 hours, 5, 6, 7, 8, 9, or about 10 hours or greater than about 3 hours, 4, 5, 6, 7, 8, 9, or about 10 hours. As a further example, settling can occur over a time in a range of from about 5 hours to about 13 hours, about 6 hours to about 10 hours, less than 7 hours, 8, 9, 10, 11, 12, or about 13 hours or greater than about 5 hours, 6, 7, 8, 9, 10, 11, 12, or about 13 hours. In some examples, settling occurs for a longer time than agitation.

After the multi-phase solution is formed, the first phase typically has a weight percent of glucose based on total carbohydrate present in the first phase in a range of from about 30 wt % to about 70 wt % , about 30 wt % to about 60 wt %, about 40 wt % to about 50 wt %, less than about 31 wt %, 32, 33, 34, 35, 36, 37, 38, 39, 40, 41, 42, 43, 44, 45, 46, 46, 48, 49, 50, 51, 52, 53, 54, 55, 56, 57, 58, 59, or about 60 or greater than about 30, 31, 32, 33, 34, 35, 36, 37, 38, 39, 40, 41, 42, 43, 44, 45, 46, 46, 48, 49, 50, 51, 52, 53, 54, 55, 56, 57, 58, 59, or about 60 wt %. To achieve the desired weight percentage of glucose, the multi-phase solution (or at least the first phase of the multi-phase solution) is typically heated to a temperature of at least about 70° C., at least about 80° C., at least about 100° C., in a range of from about 70° C. to about 130° C., about 80° C. to about 130° C., less than about 75° C., 80, 85, 90, 95, 100, 105, 110, 115, 120, 125, or about 130° C. or greater than about 70° C., 75, 80, 85, 90, 95, 100, 105, 110, 115, 120, 125, or about 130° C. Heating the multi-phase solution to these temperatures helps to deactivate the glucoamylase to prevent the first phase from having a glucose weight percentage than desired.

The second phase, by virtue of the lipids, proteins, or mixtures thereof present therein has a higher total amount of suspended solids than the first phase. A total amount of suspended solids measured in the second phase is typically at least 1000 ppm, at least 1300 ppm, at least 3000 ppm, in a range of from about 1000 ppm to about 5000 ppm, about 2500 ppm to about 3500 ppm or greater than about 1000 ppm, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or about 5000 ppm. The total suspended solids values can be measured using an inline turbidity meter. Conversely, the first phase has a lower total suspended solids number than the second phase. For example, the first phase has a total suspended solids number of less than 3000 ppm, less than 2000 ppm, less than 1000 ppm, less than 500 ppm, less than 100 ppm, less than 50 ppm, in a range of from about 1 ppm to about 3000 ppm, or about 50 ppm to about 500 ppm. The total suspended solids number determined by the inline turbidity meter is calibrated using a CEM AVC-80 microwave analyzer oven.

Using the microwave analyzer, a sample of the hydrolysate is pulled from a pipe that is downstream of the container in which it is located. That portion is contacted with a filter paper. The sample and filter paper are placed in contact with a Buchner funnel atop a 1000 mL vacuum flask connected to an aspirator. 250 mL water is aspirated through the filter paper. Retained solid is placed in the microwave analyzer over to obtain a final weight value. The total suspended solids amount is calculated by multiplying the final weight by 1,000,000 and dividing the product by the volume (mL) sample that was initially collected. This method can be used to calculate the amount of total suspended solids at any point during the process and can be used to calibrate an inline turbidity meter for expedited determination of the total suspended solids number.

After there is sufficient separation between the first phase and the second phase, the phases are isolated from each other. Isolation typically occurs for example, by draining the first phase from the bottom of the container. Draining leaves the second phase in the container. The second phase can be discarded, used as a potential feedstock, or sent to an ethanol fermentation plant, or the remaining starch hydrolysate located therein can be used to produce high dextrose equivalent solutions that can be used in turn to produce ethanol or even used to create a solution having a high dextrose equivalent number of at least 95. Typically, the second phase can have a concentration of glucose that is substantially identical to that of the first phase. However once the first phase is heat treated, the concentration of glucose in the second phase can continue to increase. Alternatively, the second phase can be repurposed as animal feed. In various examples, isolation does not include filtering the first phase from the second phase.

The separation between the first phase and the second phase can be clear or there may be a gradient such that there may not be a distinct interface between the two phases. A way to monitor whether any of the second phase is being isolated along with the first phase is to measure the total amount suspended solids of the solution as it is drained from the container. The inline turbidity meter device (e.g., an OPTEK CONTROL 4000) for measuring this is typically located at or slightly downstream of the outlet in the container. Isolation is stopped to minimize the flow of a solution having a total suspended solids amount of at least 2000 ppm, at least 3000 ppm, in a range of from about 1000 ppm to about 5000 ppm, about 1300 ppm to about 1500 ppm or greater than about 1000 ppm, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or about 5000 ppm. Total amounts of dissolved solids such as these suggest that the second phase may be present. Avoiding mixing a substantial amount of the second phase with the first phase has several benefits including limiting the amount of arabitol and other unintended by-products that are formed with the fermentation product, this can also help to prevent excessive foaming issues in the fermenter, or it can help generally to prevent fouling issues in the fermenter or components thereof (e.g., pipes and heat exchangers).

The first phase can be stored for any suitable amount of time to be used as a component of a fermentation broth. This can allow the first phase to be used for applications where the facility for carrying out fermentation is some distance away from the facility where the first phase is made or when it is not desired for fermentation to be immediately conducted. Additionally, because the glucose weight percentage of the first phase is controlled to be at a moderate level, the formation of undesirable side products such as isomaltose is substantially mitigated. For example, the amount of isomaltose in the first phase typically less than about 1.0, 0.75, 0.50, 0.10, or 0.05 g/L of isomaltose. The low amount of isomaltose can make it unnecessary to have to add potentially expensive enzymes such as a trans-glucosidase to the first phase, or subsequently to the fermentation broth, to break down undesired isomaltose.

After the first phase is isolated, at least a first portion of the first phase is added to other components to form a fermentation media or broth, which typically includes water and nutrients, such as proteins (or other nitrogen source), vitamins and salts, which are necessary or desirable for cell function. Other components may also be present in the fermentation broth or media, such as buffering agents, fermentation products (which tend to accumulate as the fermentation progresses), and other metabolites. In cases where the fermentation product is an organic acid, it is common to buffer the broth with a base such as calcium hydroxide or calcium carbonate, ammonia or ammonium hydroxide, sodium hydroxide, or potassium hydroxide in order to maintain a pH at which the microorganism functions well. For example, the pH is typically maintained in a range of from about 2.0 to about 7.0, about 3.0 to about 6.0, about 3.5 to about 4.5, about 2.5 to about 3.5, about 3.0 to 4.0, less than about 2.0, 2.5, 3.0, 3.5, 4.0, 4.5, 5.0, 5.5, 6.0, 6.7, or about 7 or greater than about 2.0, 2.5, 3.0, 3.5, 4.0, 4.5, 5.0, 5.5, 6.0, 6.7, or about 7.0

The microorganism used in the fermentation method is one which can ferment glucose to the desired fermentation product. Examples of suitable fermentation products can include an organic acid or an amino acid, a derivative thereof, or a salt thereof. Suitable examples of organic acids and amino acids can include lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof. In some examples, the fermentation method produces less than about 3 g/L of ethanol or no ethanol. In some examples, where ethanol is produced, the production can occur through contamination of the fermentation broth (e.g., growth of an undesirable microorganism).

Suitable examples of microorganisms that can be used in the fermentation method include a fungus, a bacteria, or a mixture thereof. The fungus can include a filamentous fungus or a yeast. If a yeast is used it is preferable that it is as Crabtree negative yeast. However, examples of suitable yeasts typically include Saccharomyces cerevisiae, Kluyveromyces lactis, Kluyveromyces marxianus, Yarrowia lipolytica, Pichia kudriavzevii, Schizosaccharomyces pombe, or a mixture thereof. Suitable examples of bacteria typically include Streptococcus, Lactobacillus, Bacillus, Escherichia, Salmonella, Neisseria, Acetobactor, Arthrobacter, Aspergillus, Bifdobacterium, Corynebacterium, Pseudomanas, or a mixture thereof.

The particular microorganism used is selected in relation to the fermentation product that is desired. The microorganism may be naturally occurring, or may be a mutant or recombinant strain. A recombinant strain typically includes genes or polypeptides that are endogenous, heterologous, exogenous, or a combination thereof. “Endogenous” as used herein refers to a genetic material such as a gene, a promoter and a terminator is “endogenous” to a cell if it is (i) native to the cell, (ii) present at the same location as that genetic material is present in the wild-type cell and (iii) under the regulatory control of its native promoter and its native terminator. The term “heterologous” refers to a molecule (e.g., polypeptide or nucleic acid) or activity that is from a source that is different than the referenced organism or, where present, a referenced molecule. Accordingly, a gene or protein that is heterologous to a referenced organism is a gene or protein not found in the native form of that organism. For example, a specific glucoamylase (GA) gene found in a first fungal species and exogenously introduced into a second fungal species that is the host organism is “heterologous” to the second fungal organism. As another example, a specific glucoamylase gene from a fungal species that is modified from its native form with one or more nucleotide changes that affect the function of the gene is “heterologous”. Microorganisms can also have genes deleted. For example, some microorganisms can be genetically modified by disrupting or deleting the native pyruvate decarboxylase (PDC) gene to eliminate ethanol production. This yeast can be designated as a “PDC negative engineered yeast” or a “PDC negative yeast.” All heterologous nucleic acids are also exogenous. For purposes of this application, genetic material such as genes, promoters and terminators is “exogenous” to a cell if it is (i) non-native to the cell and/or (ii) is native to the cell, but is present at a location different than where that genetic material is present in the wild-type cell and/or (iii) is under the regulatory control of a non-native promoter and/or non-native terminator. Extra copies of native genetic material are considered as “exogenous” for purposes of this invention, even if such extra copies are present at the same locus as that genetic material is present in the wild-type host strain. “Native” as used herein with regard to a metabolic pathway refers to a metabolic pathway that exists and is active in the wild-type host strain. Genetic material such as genes, promoters and terminators is “native” for purposes of this application if the genetic material has a sequence identical to (apart from individual-to-individual mutations which do not affect function) a genetic component that is present in the genome of the wild-type host cell (e.g., the exogenous genetic component is identical to an endogenous genetic component).”

An exogenous genetic component may have either a native or non-native sequence. An exogenous genetic component with a native sequence comprises a sequence identical to a genetic component that is present in the genome of a native cell (e.g., the exogenous genetic component is identical to an endogenous genetic component). However, the exogenous component is present at a different location in the host cell genome than the endogenous component. For example, an exogenous gene that is identical to an endogenous gene may be inserted into a yeast cell, resulting in a modified cell with a non-native (increased) number of gene copies. An exogenous genetic component with a non-native sequence comprises a sequence that is not found in the genome of a native cell. For example, an exogenous gene from a particular species may be inserted into a yeast cell of another species. An exogenous gene is integrated into the host cell genome in a functional manner, meaning that it is capable of producing an active protein in the host cell. However, in various examples the exogenous gene may be introduced into the cell as part of a vector that is stably maintained in the host cytoplasm. In other examples the exogenous genetic component can be in a native location but can have a modification to its promoter or terminator. In some examples, an engineered microorganism may be used in the fermentation method that is not capable of producing ethanol or at least produces a minimal amount of ethanol for example, less than about 3 g/L of ethanol.

In the fermentation method, the first phase is optionally added to the fermentation broth at least two separate times (e.g., a first portion of the first phase is added to the fermentation broth followed by adding a second portion of the fermentation broth). The fermentation broth is typically located in a container such as a fermentation vessel. In this manner the fermentation method may be a batch-fed fermentation. Additionally, glucoamylase is added to the fermentation broth. A first portion of the first phase is added before fermentation is conducted to reach the desired glucose concentration. The first portion of the first phase can include a concentration of glucose in the fermentation broth that is typically in a range of from about 55 g/L to about 95 g/L (for example in a fed-batch fermentation it can be in a range of from about 55 g/L to about 95 g/L or about 60 g/L to about 70 g/L and in a batch fermentation it can be in a range of from about 60 g/L to about 95 g/L or about 70 g/L to about 80 g/L. The fermentation broth can include water with glucose and other components described herein that are fermented in the fermentation broth in an initial fermentation step to produce a fermentation product until the fermentation broth contains about 50 g/L or less of glucose, 40 g/L or less of glucose, about 30 g/L or less of glucose, about 15 g/L or less of glucose, about 10 g/L or less of glucose, about 1 g/L to about 40 g/L of glucose, about 1 g/L to about 30 g/L of glucose, or about 15 g/L to about 30 g/L of glucose.

With respect to the amount of glucose present in the first phase that is added to the fermentation broth, it is found that a minimum initial amount of glucose is desirable in the broth to assure that the fermentation process can be initiated and continue to operate without interruption during the fermentation process. However, this amount is lower than what is typically used in a fermentation process using a saccharide component having a dextrose equivalent number of at least 95 as feedstock. Furthermore, maintaining the comparatively lower initial concentration of glucose can help to prevent contamination in the fermentation broth. This is because having a relatively limited amount of glucose available can limit the initial grown in the culture of the microorganism. With the growth of the microorganism controlled it is less likely that a species of microorganism, other than the intended microorganism, will be grown in any significant number. This in turn, helps to limit the product of unintended or unwanted fermentation products.

After the first portion of the first phase is added to the fermentation broth a second portion of the first phase is added. Fermentation continues before the second portion is added such that the amount of glucose present (g/L) is decreased. Although the amount of glucose present can increase (e.g., to 50 g/L or higher) at certain points during fermentation. The second portion of the first phase is added is such a manner to maintain a concentration of glucose in a range of from about 1 g/L to about 25 g/L, about 2 g/L to about 20 g/L, about 5 g/L to about 25 g/L, about 8 g/L to about 12 g/L, about 13 g/L to about 17 g/L, in the fermentation broth, less than about, 2 g/L, 3, 4, 5, 6, 7, 8, 9, 10, 11, 12, 13, 14, 15, 16, 17, 18, 19, 20, 21, 22, 23, 24, or about 25 g/L or greater than about 1 g/L, 2, 3, 4, 5, 6, 7, 8, 9, 10, 11, 12, 13, 14, 15, 16, 17, 18, 19, 20, 21, or about 22 g/L.

The second portion of the first phase can be added to the first portion that was generated in the same partial saccharification process. However, in some examples, it is possible for the first portion of the first phase from one partial saccharification process to be added to the second portion of a first phase from another partial saccharification process.

The fermentation is carried out under conditions tailored to the microorganism and desired fermentation product. Although conditions can vary depending on the particular microorganism and desired fermentation product, suitable conditions typically include a temperature of from about 20° C., of from about 30° C. to about 50° C., less than about 20° C., 25, 30, 35, 40, 45, or about 50° C. or greater than about 20° C., 25, 30, 35, 40, 45, or about 50° C. Usually the fermentation broth is mixed. The mixing may occur by sparging gas to the fermentation broth or alternatively via direct mechanical agitation or by other means.

The first portion of the first phase, second portion of the first phase, or both can be added to the fermentation broth using a variable rate addition system. This system can be desirable because it does not introduce temporarily high concentrations of glucose in the fermentation batch that would adversely affect the activity of the enzyme. Examples of such systems include a variable speed pump or a metering valve (such as a throttle valve) operably connected to a pump, which pump or valve can be utilized to vary the amount of hydrolysate introduced into the fermentation broth over time. In an example, the starch hydrolysate addition is carried out over a time period of from about 4 to about 30 hours, for example from about 5 to about 24 hours, and in some cases to optimize product yield, enzyme usage and production costs, from about 10 to about 20 hours.

In some examples, the fermentation method can carried out in aerobic, microaerobic or anaerobic conditions. By “microaerobic” it is meant that some oxygen is fed to the fermentation, and the microorganisms take up the oxygen fast enough such that the dissolved oxygen concentration averages less than about 2% of the saturated oxygen concentration under atmospheric air for at least five hours of the fermentation. Also, the average oxygen transfer rate of a microaerobic fermentation is typically in a range of from about 3 mmol L⁻¹h⁻¹ to about 80 mmol L⁻¹ h⁻¹, about 10 mmol L⁻¹ h⁻¹ to about 60 mmol L⁻¹ h⁻¹, about 25 to about 45 mmol L⁻¹ h⁻¹, less than about 3 mmol L⁻¹ h⁻¹, 5, 10, 15, 20, 25, 30, 35, 40, 45, 50, 55, 60, 65, 70, 75, or about 80 mmol L⁻¹ h⁻¹ or greater than about 3 mmol L⁻¹ h⁻¹, 5, 10, 15, 20, 25, 30, 35, 40, 45, 50, 55, 60, 65, 70, 75, or about 80 mmol L⁻¹ h⁻¹. According to various examples, the oxygen transfer rate in the method can be proportional to the rate of production of any organic acid, amino acid, derivative thereof, or salt thereof described herein.

In an example, during the addition of the first portion of the first phase, second portion of the first phase, or both starch hydrolysate the glucose concentration is monitored by a real-time monitoring system. Real-time monitoring systems include systems that directly monitor glucose concentration and systems that indirectly monitor glucose concentration. Examples of real-time monitoring systems that typically directly monitor glucose concentration include systems based on infrared (IR) spectroscopy, near-infrared (NIR) spectroscopy systems, Fourier transform infrared (FTIR) systems, systems based on refractive index, automated enzyme based measurement systems such as a YSI 2950 Biochemistry Analyzer sold by YSI Life Sciences systems, high performance liquid chromatography (HPLC) based systems, gas chromatography (GC) based systems, and other real-time monitoring systems known to one of skill in the art.

Additionally real-time monitoring systems that indirectly monitor/measure the glucose concentration of a fermentation process can be developed by determining the typical carbon distribution in a particular fermentation process and correlating the glucose concentration present in the fermentation broth to another parameter exhibited by the fermentation, such as, for example, a correlation of the glucose level present in the fermentation broth with a measurement of the carbon dioxide evolution rate and the amount of carbon dioxide present in an off-gas stream from the fermentation vessel. The carbon dioxide can be readily measured through use of a mass spectrometer or other suitable instrumental technique for measuring the components of the off-gas stream. In an example, the glucose concentration is monitored by a real-time monitoring system using infrared spectroscopy. In another example, the glucose concentration is monitored by a real-time monitoring system using near-infrared spectroscopy. In one example, the real time monitoring systems interface with equipment that controls the introduction of starch hydrolysate introduced into the fermentation broth to facilitate the maintenance of the glucose concentration in the fermentation broth at the desired concentration. In another example, the glucose concentration is monitored by real-time monitoring system, and a control system utilizes the value of glucose concentration measured to control the introduction of starch hydrolysate (and optionally enzyme) into the fermentation broth.

After the second portion of the first phase, as well as any additional portions of the first phase are added to the fermentation broth, the fermentation can be allowed to proceed until the final fermentation broth is produced. In an example, after the desired amount of the first phase has been added, the fermentation is allowed to proceed until the glucose concentration is at a concentration below 5 g/L, or below 1 g/L, or below 0.5 g/L based on the volume of the final fermentation broth.

There are various recovery methods that can be used to retrieve the fermentation product. When the fermentation process utilizes a gypsum recovery process, as described below, a suitable cation utilized includes calcium. In a gypsum recovery process, an organic acid fermentation product (including a hydroxy carboxylic acid) is made. During the fermentation, the pH of the fermentation broth is maintained at a desirable level using calcium hydroxide, often used in the form of lime. Once the fermentation is complete, it can be desirable to enhance the recovery of the organic acid by adding sulfuric acid to acidulate the broth and thereby increase the percentage of acid that is recovered from the broth. The ions from the lime and sulfuric acid react to form calcium sulfate (gypsum), which can then be readily removed from the fermentation broth by precipitation.

Alternatively, ammonia or ammonium hydroxide is economical and abundant. Ammonium hydroxide has the advantage of providing not only pH buffering, but also supplies an additional bio-available nitrogen source to the fermentation. Examples of fermentations that employ ammonium hydroxide for pH control include fermentation processes for the manufacture of citric acid, lysine or other amino acids. Additionally, ammonium hydroxide is commonly employed in fermentation processes that typically incorporate the ammonium into the fermentation product (such as amino acids) or biomass (such as amino acids or citric acid and other processes known to one of skill in the art), can tolerate ammonium in the final product specifications or processes where the fermentation product is typically recovered through the use of ion exchange, crystallization or liquid-liquid extraction (such as citric acid, succinic acid, or fumaric acid, and other processes known to one of skill art).

Sodium hydroxide and potassium hydroxide are readily available. They typically would be utilized in fermentation processes that require only relatively minor pH control during the fermentation.

The fermentation product is recovered from the fermentation broth. The manner of accomplishing this will depend on the particular product. However, in general, the microorganism is separated from the liquid phase, typically via a filtration step or centrifugation step, and the product recovered via, for example, distillation, extraction, crystallization, membrane separation, osmosis, reverse osmosis, or other suitable technique. Organic acids typically will require that the fluid containing the organic acid (and salts thereof) be acidulated using acids such as sulfuric acid to recover the organic acid.

The present process provides the ability to make fermentation products on a production scale level with excellent yields and purity. In an example, the batch process is carried out in to produce batches of at least 25,000 gallons of final fermentation broth.

### WORKING EXAMPLES

Various examples of the present invention can be better understood by reference to the following Examples which are offered by way of illustration. The present invention is not limited to the Examples given herein.

### Working Example 1: Hydrolysate Preparation

A specific glucoamylase enzyme product is not required for this process. The Working Example described uses Extenda Go, available from Novozymes, Bagsvaerd Denmark, in the saccharification step and Distillase SSF, available from DuPont INC, Wilmington, Del. in the fermentation step.

The starch hydrolysate includes a starch slurry, in this case corn starch from Cargill's Wet Corn Mill in Blair Nebraska. Through a process called liquefaction, the starch slurry is treated with alpha-amylase enzymes and heat and is then given time to liquefy the starch slurry into an 8-13 Dextrose Equivalent (D.E.) hydrolysate.

The liquefied hydrolysate is cooled and pH adjusted inline as it is transferred to separate tanks for process called saccharification. After cooling and pH adjustment is completed, the first glucoamylase enzyme is added, the optimal temperature and pH is enzyme dependent. The actual amount of enzyme added will be dependent on equipment sizing and the activity of the enzyme itself, in this example 0.025% (volume of enzyme/volume of saccharification tank). However, the saccharification step for the described process carried out for a minimum 13 hours, with 15 hours showing good performance. Once the glucoamylase is added and the saccharification tank is filled completely the material is agitated for 4 hours and then the agitation is stopped. During this time the glucose concentration in the reaction increases to approximately 15-25 wt %, based on total carbohydrate present. Without agitation, the glucose liberation continues until it is between 40-60 wt %, based on total carbohydrate present. The reaction is considered complete when the total reaction time is greater than 13 hours and the glucose concentration is between 40-60 wt %, based on total carbohydrate present, the material should not be used in the fermentation process if it has converted over 80 wt % glucose.

Once the minimum requirements of 4 hours of agitation followed by 9-11 hours of settling, 13-15 hours total, of reaction time and 40-60 wt % glucose, based on total carbohydrate present have been met, the partially saccharified hydrolysate (fully saccharified typically implies >95% wt % glucose, based on total carbohydrate present) is transferred to storage. During the period of saccharification without agitation the insoluble solids in the tank float towards the top of the vessel. The transfer to storage proceeds through the bottom of the vessel in a manner that does not disturb the upper levels of the mixture. During the transfer the material is monitored with an inline turbidity meter. The turbidity measurement is correlated to total suspended solids. Once the measured total suspended solids reaches 3000 ppm the transfer to starch hydrolysate storage is stopped and the remaining material in the saccharification tank is directed to an alternative storage tank where it is metered into the facilities ethanol fermentation process. The total suspended solids amount is measured as described above with respect to the inline turbidity meter. The material that goes forward to storage is heated to at least 75° C. inline before it reaches the storage tank in order to prevent further conversion by deactivating the glucoamylase. At this point the starch hydrolysate can be stored or transported in a similar manner to any commercial corn syrup product.

### Working Example 2: Fed-Batch Fermentation Process

The fermentation broth includes water as well as sufficient nutrients required to enable cell growth. The fermentation broth also contains an anti-foam agent to control foaming in the fermentation.

A fermenter having a capacity of about 1,000,000 L (about 290,000 gallons) is partially filled to 72% of volumetric operating capacity with the components of the fermentation broth as well as 70% of the total hydrolysate to be added. The glucose source for cell growth and fermentation is the starch hydrolysate described in Working Example 1. The temperature is controlled at a value that is suitable for the fermentation host organism, in this case 34° C. The fermenter is then inoculated with a suitable fermentation microorganism, in this case, a yeast engineered for the production of lactic acid. During fermentation, the pH is maintained above 3.1 by the addition of calcium hydroxide.

As the fermentation proceeds, the glucose is converted into lactic acid at a certain yield. The glucose concentration is monitored by a near infrared spectroscopy system (NIR) (Brueker—Matrix F model). When the glucose is reduced to less than 40 g/L as measured by NIR, the second glucoamylase enzyme as described above is added at a dose of 0.0.36-0.032% (volume enzyme solution/volume fermentation broth). This concentration of glucoamylase enzyme is sufficient for a 48 hour fermentation. Longer batches will require less glucoamylase enzyme, shorter batches require more glucoamylase enzyme.

The fermentation is then allowed to further proceed until the glucose concentration reaches 10 g/L. The remaining 30% of the total hydrolysate is then gradually added to the fermenter at a rate controlled to maintain the glucose between 8 g/L and 12 g/L as measured by the NIR.

Once 100% of the target dextrose has been added, the hydrolysate feed is stopped and the fermentation proceeds until the glucose is reduced below 0.3 g/L. As measured by a properly calibrated Shodex SZ5532 Sugar HPLC method the remaining carbohydrate profile is approximately 0.3 g/L Isomaltose, 0.2 g/l Maltose, and 0.12 g/L Panose.

### Working Example 3: Batch Fermentation Process

The fermentation broth includes water as well as sufficient nutrients required to enable cell growth. The fermentation broth also contains an anti-foam agent to control foaming in the fermentation.

A fermenter is partially filled to 81% of volumetric operating capacity with the components of the fermentation broth as well as 100% of the total hydrolysate to be added. The glucose source for cell growth and fermentation is the starch hydrolysate described in Example 1. The temperature is controlled at a value that is suitable for the fermentation host organism, in this case 34° C. The fermenter is then inoculated with a suitable fermentation microorganism, in this case, a yeast engineered for the production of lactic acid. The pH is maintained above 3.1 by the addition of calcium hydroxide.

As the fermentation proceeds, the glucose is converted into lactic acid at a certain yield. The glucose concentration is monitored by a near infrared spectroscopy system (NIR) (Brueker—Matrix F model). When the glucose has been reduced to less than 40 g/L as measured by NIR, the second glucoamylase enzyme as described above is added at a dose of 0.032%-0.36% (volume enzyme solution/volume fermentation broth). This concentration of enzyme is sufficient for a 48 hour fermentation. Longer batches will require less, shorter batches more.

The fermentation proceeds until the glucose is reduced below 0.3 g/L. As measured by a properly calibrated Shodex SZ5532 Sugar HPLC method the remaining carbohydrate profile is approximately 0.3 g/L Isomaltose, 0.2 g/l Maltose, and 0.12 g/L Panose.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present invention. Thus, it should be understood that although the present invention has been specifically disclosed by specific examples and optional features, modification and variation of the concepts herein disclosed may be resorted to by those of ordinary skill in the art, and that such modifications and variations are considered to be within the scope of examples of the present invention.

### Working Example 4: Hydrolysate Preparation

A specific glucoamylase enzyme product is not required for this process. The Working Example described uses Extenda Go, available from Novozymes, Bagsvaerd Denmark, in the saccharification step and Distillase SSF, available from DuPont INC, Wilmington, Del. in the fermentation step.

The starch hydrolysate includes a starch slurry, in this case corn starch from Cargill's Wet Corn Mill in Blair Neb. Through a process called liquefaction, the starch slurry is treated with alpha-amylase enzymes and heat and is then given time to liquefy the starch slurry into an 8-13 Dextrose Equivalent (D.E.) hydrolysate.

The liquefied hydrolysate is cooled and pH adjusted inline as it is transferred to separate tanks for process called saccharification. In this example the hydrolysate is cooled to between 68-71° C. After cooling and pH adjustment is completed, the first glucoamylase enzyme is added. The actual amount of enzyme added will be dependent on equipment sizing and the activity of the enzyme itself, in this example 0.025% (volume of enzyme/volume of saccharification tank). However, the saccharification step for the described process carried out for a minimum 13 hours, with 15 hours showing good performance. Once the glucoamylase is added and the saccharification tank is filled completely the material is agitated for 4 hours and then the agitation is stopped. During this time the glucose concentration in the reaction increases to approximately 15-25 wt %, based on total carbohydrate present. Without agitation, the glucose liberation continues until it is between 40-60 wt %, based on total carbohydrate present. The reaction is considered complete when the total reaction time is greater than 13 hours and the glucose concentration is between 40-60 wt %, based on total carbohydrate present. Using a 68-71° C. temperature range during this step helps to prevent the conversion reaction from reaching an undesired >80 wt % dextrose concentration.

Once the minimum requirements of 4 hours of agitation followed by 9-11 hours of settling, 13-15 hours total, of reaction time and 40-60 wt % glucose, based on total carbohydrate present have been met, the partially saccharified hydrolysate (fully saccharified typically implies >95% wt % glucose, based on total carbohydrate present) is transferred to storage. During the period of saccharification without agitation the insoluble solids in the tank float towards the top of the vessel. The transfer to storage proceeds through the bottom of the vessel in a manner that does not disturb the upper levels of the mixture. During the transfer the material is monitored with an inline turbidity meter. The turbidity measurement is correlated to total suspended solids. Once the measured total suspended solids reaches 3000 ppm the transfer to starch hydrolysate storage is stopped and the remaining material in the saccharification tank is directed to an alternative storage tank where it is metered into the facilities ethanol fermentation process. The total suspended solids amount is measured as described above with respect to the inline turbidity meter. The material that goes forward to storage is heated to at least 75° C. inline before it reaches the storage tank in order to prevent further conversion by deactivating the glucoamylase. At this point the starch hydrolysate can be stored or transported in a similar manner to any commercial corn syrup product.

### Working Example 5: Fed-Batch Fermentation Process

The fermentation broth includes water as well as sufficient nutrients required to enable cell growth. The fermentation broth also contains an anti-foam agent to control foaming in the fermentation.

A fermenter having a capacity of about 1,000,000 L (about 290,000 gallons) is partially filled to 72% of volumetric operating capacity with the components of the fermentation broth as well as 70% of the total hydrolysate to be added. The glucose source for cell growth and fermentation is the starch hydrolysate described in Working Example 1. The temperature is controlled at a value that is suitable for the fermentation host organism, in this case 34° C. The fermenter is then inoculated with a suitable fermentation microorganism, in this case, a yeast engineered for the production of lactic acid. As the fermentation proceeds, the pH is maintained from 4 to 5 (for example from 4.2 to 4.6, from 4.3 to 4.5), and typically at a pH of about 4.4 by the addition of calcium hydroxide. Once a sufficient amount of lime has been added the fermentation pH is allowed to free fall to the desired final pH, in this example 3.1. The amount of lime added is determined by the expected lactic concentration at the end of fermentation and the level of free acid desired.

As the fermentation proceeds, the glucose is converted into lactic acid at a certain yield. The glucose concentration is monitored by a near infrared spectroscopy system (NIR) (Brueker—Matrix F model). When the glucose is reduced to less than 40 g/L as measured by NIR, the second glucoamylase enzyme as described above is added at a dose of 0.0.36-0.032% (volume enzyme solution/volume fermentation broth). The enzyme is added while the pH is still under control at 4.4 pH. This concentration of glucoamylase enzyme is sufficient for a 48 hour fermentation. Longer batches will require less glucoamylase enzyme, shorter batches require more glucoamylase enzyme.

The fermentation is then allowed to further proceed until the glucose concentration reaches 10 g/L. The remaining 30% of the total hydrolysate is then gradually added to the fermenter at a rate controlled to maintain the glucose between 8 g/L and 12 g/L as measured by the NIR.

Once 100% of the target dextrose has been added, the hydrolysate feed is stopped and the fermentation proceeds until the glucose is reduced below 0.3 g/L. As measured by a properly calibrated Shodex SZ5532 Sugar HPLC method the remaining carbohydrate profile is approximately 0.3 g/L Isomaltose, 0.2 g/l Maltose, and 0.12 g/L Panose.

### EXEMPLARY EXAMPLES

The following exemplary examples are provided, the numbering of which is not to be construed as designating levels of importance:

Example 1 provides a fermentation process, the process comprising:

(a) contacting a starch hydrolysate with a glucoamylase with agitation;

(b) ceasing the agitation to allow for settling to form a multi-phase solution, a first phase of the multi-phase solution comprising a saccharide component comprising about 30 wt % to about 80 wt % (for example 30 wt % to 70 wt %, 30 wt % to 60 wt %) based on a total carbohydrate present in the first and a second phase of the multi-phase solution comprising a higher amount of total suspended solids than the first phase;

(c) draining the first phase to isolate the first phase from the second phase to form a fermentation broth comprising a first portion of the first phase;

(d) heating at least the first phase to a temperature of at least 70° C.;

(e) fermenting the fermentation broth with a microorganism to produce a fermentation product in the fermentation broth until a concentration of glucose in the fermentation broth is 40 g/L or less;

(f) contacting the fermentation broth with glucoamylase; and

(g) adding a second portion of the first phase to the fermentation broth to maintain a concentration of glucose in a range of from about 1 g/L to about 20 g/L in the fermentation broth.

Example 2 provides the fermentation process of Example 1, further comprising:

(h) contacting the starch hydrolysate with an alpha-amylase before contacting the starch hydrolysate with the glucoamylase according to (a).

Example 3 provides the fermentation process of Example 1, wherein after (a) a solution including the starch hydrolysate has a dextrose equivalent number in a range of from about 1 to about 15.

Example 4 provides the fermentation process of any one of Examples 1 or 2, wherein after (a) a solution including the starch hydrolysate has a dextrose equivalent number in a range of from about 8 to about 13.

Example 5 provides the fermentation process of any one of Examples 1-4, wherein contacting the starch hydrolysate with the glucoamylase with agitation at (a) further comprises heating the starch hydrolysate to a temperature of at least 80° C.

Example 6 provides the fermentation process of any one of Examples 1-5, wherein contacting the starch hydrolysate with the glucoamylase with agitation at (a) further comprises heating the starch hydrolysate to a temperature of at least 100° C.

Example 7 provides the fermentation process of any one of Examples 1-6, wherein contacting the starch hydrolysate with the glucoamylase with agitation at (a) further comprises heating the starch hydrolysate to a temperature in a range of from about 80° C. to about 130° C.

Example 8 provides the fermentation process of any one of Examples 1-7, wherein the agitation at (a) comprises stirring.

Example 9 provides the fermentation process of any one of Examples 1-8, wherein the agitation at (a) is performed for a time in a range of from about 3 hours to about 10 hours.

Example 10 provides the fermentation process of any one of Examples 1-9, wherein the agitation at (a) is performed for a time in a range of from about 5 hours to about 8 hours.

Example 11 provides the fermentation process of any one of Examples 1-10, wherein the settling at (b) is performed for a time in a range of from about 5 hours to about 13 hours.

Example 12 provides the fermentation process of any one of Examples 1-11, wherein the settling at (b) is performed for a time in a range of from about 6 hours to about 10 hours.

Example 13 provides the fermentation process of any one of Examples 1-12, wherein the settling at (b) is performed for a longer amount of time than agitation.

Example 14 provides the fermentation process of any one of Examples 1-13, wherein the second phase comprises proteins, lipids, or a mixture thereof.

Example 15 provides the fermentation process of any one of Examples 1-14, wherein adding a second portion of the first phase to the fermentation broth at (g) occurs as the fermentation product is produced.

Example 16 provides the fermentation process of any one of Examples 1-15, wherein a total amount of the suspended solids in the second phase is at least 1000 ppm.

Example 17 provides the fermentation process of any one of Examples 1-16, wherein a total suspended solids amount of the second phase is at least 3000 ppm.

Example 18 provides the fermentation process of any one of Examples 1-17, wherein a total suspended solids amount of the first phase is less than about 3000 ppm.

Example 19 provides the fermentation process of any one of Examples 1-18, wherein a total suspended solids amount of the first phase is less than about 1000 ppm.

Example 20 provides the fermentation process of any one of Examples 1-19, wherein a total suspended solids amount of the first phase is less than a total suspended solids amount of the second phase.

Example 21 provides the fermentation process of any one of Examples 1-20, wherein draining is ceased at (c) when a total amount of the suspended solids measured in a pipe downstream of a tank including the multi-phase solution is at least 1000 ppm.

Example 22 provides the fermentation process of any one of Examples 1-21, wherein draining is ceased at (c) when a total amount of the suspended solids measured in a pipe downstream of a tank including the multi-phase solution is at least 1300 ppm.

Example 23 provides the fermentation process of any one of Examples 1-22, wherein draining is ceased at (c) when a total amount of the suspended solids measured in a pipe downstream of a tank including the multi-phase solution is in a range of from about 1000 ppm to about 5000 ppm.

Example 24 provides the fermentation process of any one of Examples 1-23, wherein draining is ceased at (c) when a total amount of the suspended solids measured in a pipe downstream of a tank including the multi-phase solution is in a range of from about 1300 ppm to about 1500 ppm.

Example 25 provides the fermentation process of any one of Examples 1-24, wherein draining is ceased at (c) at a point to where the first phase comprises a total suspended solids amount of less than 3000 ppm.

Example 26 provides the fermentation process of any one of Examples 1-25, wherein the saccharide component comprises glucose.

Example 27 provides the fermentation process of any one of Examples 1-26, wherein the heating of the first phase to at least 70° C. at (d) comprises heating the first phase to at least 80° C.

Example 28 provides the fermentation process of any one of Examples 1-27, wherein heating of the first phase to at least 70° C. at (d) comprises heating to a temperature in a range of from about 80° C. to about 130° C.

Example 29 provides the fermentation process of any one of Examples 1-28, wherein the draining of the first phase to isolate the first phase from the second phase to form the fermentation broth at (c) comprises draining the first phase from a bottom of a tank.

Example 30 provides the fermentation process of any one of Examples 1-29, further comprising:

(i) collecting the second phase for use as animal feed, collecting the second phase feedstock for an ethanol fermentation plant, processing the second phase to create a solution having a dextrose equivalent number of at least 95, or a combination thereof after (c).

Example 31 provides the fermentation process of any one of Examples 1-30, wherein the microorganism comprises a fungus, a bacterium, or a mixture thereof.

Example 32 provides the fermentation process of Example 31, wherein the fungus comprises a yeast.

Example 33 provides the fermentation process of Example 32, wherein the yeast comprises Saccharomyces cerevisiae, Kluyveromyces lactis, Kluyveromyces marxianus, Yarrowia lipolytica, Pichia kudriavzevii, Schizosaccharomyces pombe, or a mixture thereof.

Example 34 provides the fermentation process of any one of Examples 31-33, wherein the bacteria comprises Streptococcus, Lactobacillus, Bacillus, Escherichia, Salmonella, Neisseria, Acetobactor, Arthrobacter, Aspergillus, Bifdobacterium, Corynebacterium, Pseudomanas, or a mixture thereof.

Example 35 provides the fermentation process of any one of Examples 1-34, wherein the microorganism is an engineered microorganism.

Example 36 provides the fermentation process of Example 35, wherein the engineered microorganism is a yeast that is PDC negative and is not capable of producing ethanol.

Example 37 provides the fermentation process of any one of Examples 1-36, wherein the fermentation broth is fermented at (e) to produce the fermentation product and wherein a concentration of the glucose in the fermentation broth is 15 g/L or less and a glucoamylase is added at (f).

Example 38 provides the fermentation process of any one of Examples 1-37, wherein the fermentation broth is fermented at (e) to produce the fermentation product and wherein a concentration of the glucose in the fermentation broth is 1 g/L or less and a glucoamylase is added at (f).

Example 39 provides the fermentation process of any one of Examples 1-38, wherein the fermentation broth is fermented at (e) to produce the fermentation product and wherein a concentration of the glucose in the fermentation broth comprising the first portion of the first phase is in a range of from about 0.1 g/L to about 40 g/L and a glucoamylase is added at (f).

Example 40 provides the fermentation process of any one of Examples 1-39, wherein the fermentation broth comprising the first portion of the first phase is fermented at (e) to produce the fermentation product and wherein a concentration of the glucose is in a range of from about 3 g/L to about 25 g/L and a glucoamylase is added at (f).

Example 41 provides the fermentation process of any one of Examples 1-40, wherein the second portion of the first phase is added at (g) to the fermentation broth to maintain a concentration of the glucose in a range of from about 8 g/L to about 12 g/L.

Example 42 provides the fermentation process of any one of Examples 1-41, wherein the fermentation product comprises less than about 3 g/L of ethanol.

Example 43 provides the fermentation process of any one of Examples 1-42, wherein the fermentation product is substantially free of ethanol.

Example 44 provides the fermentation process of any one of Examples 1-43, wherein the fermentation is stopped after (g) when the concentration of the glucose in the fermentation broth is 0.5 g/L or less.

Example 45 provides the fermentation process of any one of Examples 1-44, wherein the fermentation is stopped after (g) when the concentration of the glucose in the fermentation broth is 0.3 g/L or less.

Example 46 provides the fermentation process of any one of Examples 1-45, wherein the fermentation is stopped after (g) when the concentration of the glucose in the fermentation broth is in a range of from about 0 g/L to about 0.5 g/L.

Example 47 provides the fermentation process of any one of Examples 1-46, wherein the fermentation is stopped after (g) when the concentration of the glucose in the fermentation broth is in a range of from about 0.2 g/L to about 0.4 g/L.

Example 48 provides the fermentation process of any one of Examples 1-47, wherein the method produces less than about 1.5 g/L of isomaltose in the first phase.

Example 49 provides the fermentation process of any one of Examples 1-48, wherein the method produces less than 0.05 g/L isomaltose in the first phase.

Example 50 provides the fermentation process of any one of Examples 1-49, wherein the fermentation product comprises an organic acid or an amino acid, a derivative thereof, or a salt thereof.

Example 51 provides the fermentation process of any one of Examples 1-49, wherein fermentation product the organic acid or amino acid comprises lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof.

Example 52 provides the fermentation process of any one of Examples 1-51, wherein the method produces at least 90 g/L of fermentation product in the fermentation broth at (e), (g), or both.

Example 53 provides the fermentation process of any one of Examples 1-52, wherein the method produces at least 120 g/L of fermentation product in the fermentation broth at (e), (g), or both.

Example 54 provides the fermentation process of any one of Examples 1-53, wherein the method produces about 100 g/L to about 130 g/L of fermentation product in the fermentation broth at (e), (g), or both.

Example 55 provides the fermentation process of any one of Examples 1-54, wherein the method produces about 90 g/L to about 150 g/L of fermentation product in the fermentation broth at (e), (g), or both.

Example 56 provides the fermentation process of any one of Examples 1-55, wherein glucose concentration is monitored by a real-time monitoring system, wherein the real-time monitoring system interfaces with equipment that controls the introduction of the first portion of the first phase, the second portion of the first phase, or both.

Example 57 provides the fermentation process of any one of Examples 1-56, wherein the method is free of adding a trans-glucosidase to the first phase, fermentation broth, or both.

Example 58 provides the fermentation process of any one of Examples 1-57, further comprising:

(j) filtering the microorganism from the fermentation broth and recovering the fermentation product.

Example 59 provides the fermentation process of any one of Examples 1-58, wherein step (f) is performed when the concentration of glucose in the fermentation broth is 40 g/L or less.

Example 60 provides the fermentation process of any one of Examples 1-59, wherein steps (a)-(g) are performed in sequential order.

Example 61 provides a fermentation process, the process comprising:

(a) contacting a starch hydrolysate with an alpha-amylase before contacting the starch hydrolysate with glucoamylase, wherein the starch hydrolysate has a dextrose equivalent number in a range of from about 8 to about 13;

(b) contacting the starch hydrolysate with a glucoamylase with agitation;

(c) ceasing agitation for a sufficient amount of time to allow for settling to form a multi-phase solution, a first phase of the multi-phase solution comprising a saccharide component comprising about 30 wt % to about 80 wt % (for example 30 wt % to 70 wt %, 30 wt % to 60 wt %) based on a total carbohydrate present in the first phase and a second phase of the multi-phase solution comprising a higher total suspended solids amount than the first phase, wherein settling is performed for a time in a range of from about 5 hours to about 13 hours;

(d) draining the first phase to isolate the first phase from the second phase to form a fermentation broth comprising a first portion of the first phase;

(e) heating at least the first phase to a temperature of at least 70° C.;

(f) fermenting the fermentation broth with a PDC negative engineered yeast to produce at least 90 g/L of a fermentation product comprising lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof, wherein a concentration of glucose is 40 g/L or less;

(g) contacting the first phase with glucoamylase; and

(h) adding a second portion of the first phase to the fermentation broth to maintain a concentration of glucose in a range of from about 1 g/L to about 20 g/L.

Example 62 provides a fermentation process, the process comprising:

(a) contacting a starch hydrolysate with a glucoamylase with agitation;

(b) ceasing the agitation to allow for settling to form a multi-phase solution, a first phase of the multi-phase solution comprising a saccharide component comprising about 30 wt % to about 80 wt % (for example 30 wt % to 70 wt %, 30 wt % to 60 wt %) based on a total carbohydrate present in the first phase and a second phase of the multi-phase solution comprising a higher amount of total suspended solids than the first phase;

(c) draining the first phase to isolate the first phase from the second phase to form a fermentation broth comprising a first portion of the first phase;

(d) heating at least the first phase to a temperature of at least 70° C.;

(e) fermenting the fermentation broth with a microorganism to produce a fermentation product in the fermentation broth and wherein a concentration of glucose in the fermentation broth is 40 g/L or less; and

(f) contacting the fermentation broth with glucoamylase.

Example 63 provides the method of Example 62, further comprising:

(g) adding a second portion of the first phase to the fermentation broth to maintain a concentration of glucose in a range of from about 1 g/L to about 20 g/L in the fermentation broth.

Example 64 provides the fermentation process of any one of Examples 62 or 63, further comprising:

(h) contacting the starch hydrolysate with an alpha-amylase before contacting the starch hydrolysate with the glucoamylase according to (a).

Example 65 provides the fermentation process of Example 62, wherein after (a) a solution including the starch hydrolysate has a dextrose equivalent number in a range of from about 1 to about 15.

Example 66 provides the fermentation process of any one of Examples 62-65, wherein after (a) a solution including the starch hydrolysate has a dextrose equivalent number in a range of from about 8 to about 13.

Example 67 provides the fermentation process of any one of Examples 62-66, wherein contacting the starch hydrolysate with the glucoamylase with agitation at (a) further comprises heating the starch hydrolysate to a temperature of at least 80° C.

Example 68 provides the fermentation process of any one of Examples 62-67, wherein contacting the starch hydrolysate with the glucoamylase with agitation at (a) further comprises heating the starch hydrolysate to a temperature of at least 100° C.

Example 69 provides the fermentation process of any one of Examples 62-68, wherein contacting the starch hydrolysate with the glucoamylase with agitation at (a) further comprises heating the starch hydrolysate to a temperature in a range of from about 80° C. to about 130° C.

Example 70 provides the fermentation process of any one of Examples 62-69, wherein the agitation at (a) comprises stirring.

Example 71 provides the fermentation process of any one of Examples 62-70, wherein the at (a) agitation is performed for a time in a range of from about 3 hours to about 10 hours.

Example 72 provides the fermentation process of any one of Examples 62-71, wherein the agitation at (a) is performed for a time in a range of from about 5 hours to about 8 hours.

Example 73 provides the fermentation process of any one of Examples 62-72, wherein the settling at (b) is performed for a time in a range of from about 5 hours to about 13 hours.

Example 74 provides the fermentation process of any one of Examples 62-73, wherein the settling at (b) is performed for a time in a range of from about 6 hours to about 10 hours.

Example 75 provides the fermentation process of any one of Examples 62-74, wherein the settling at (b) is performed for a longer amount of time than agitation.

Example 76 provides the fermentation process of any one of Examples 62-75, wherein the second phase comprises proteins, lipids, or a mixture thereof.

Example 77 provides the fermentation process of any one of Examples 63-76, wherein adding a second portion of the first phase to the fermentation broth at (g) occurs as the fermentation product is produced.

Example 78 provides the fermentation process of any one of Examples 62-77, wherein a total amount of the suspended solids in the second phase is at least 1000 ppm.

Example 79 provides the fermentation process of any one of Examples 62-78, wherein a total suspended solids amount of the second phase is at least 3000 ppm.

Example 80 provides the fermentation process of any one of Examples 62-79, wherein a total suspended solids amount of the first phase is less than about 3000 ppm.

Example 81 provides the fermentation process of any one of Examples 62-80, wherein a total suspended solids amount of the first phase is less than about 1000 ppm.

Example 82 provides the fermentation process of any one of Examples 62-81, wherein a total suspended solids amount of the first phase is less than a total suspended solids amount of the second phase.

Example 83 provides the fermentation process of any one of Examples 62-82, wherein draining is ceased at (c) when a total amount of the suspended solids measured in a pipe downstream of a tank including the multi-phase solution is at least 1000 ppm.

Example 84 provides the fermentation process of any one of Examples 62-83, wherein draining is ceased at (c) when a total amount of the suspended solids measured in a pipe downstream of a tank including the multi-phase solution is at least 1300 ppm.

Example 85 provides the fermentation process of any one of Examples 62-84, wherein draining is ceased at (c) when a total amount of the suspended solids measured in a pipe downstream of a tank including the multi-phase solution is in a range of from about 1000 ppm to about 5000 ppm.

Example 86 provides the fermentation process of any one of Examples 62-85, wherein draining is ceased at (c) when a total amount of the suspended solids measured in a pipe downstream of a tank including the multi-phase solution is in a range of from about 1300 ppm to about 1500 ppm.

Example 87 provides the fermentation process of any one of Examples 62-86, wherein draining is ceased at (c) at a point to where the first phase comprises a total suspended solids amount of less than 3000 ppm.

Example 88 provides the fermentation process of any one of Examples 62-87, wherein the saccharide component comprises glucose.

Example 89 provides the fermentation process of any one of Examples 62-88, wherein the heating of the first phase to at least 70° C. at (d) comprises heating the first phase to at least 80° C.

Example 90 provides the fermentation process of any one of Examples 62-89, wherein heating of the first phase to at least 70° C. at (d) comprises heating to a temperature in a range of from about 80° C. to about 130° C.

Example 91 provides the fermentation process of any one of Examples 62-90, wherein the draining of the first phase to isolate the first phase from the second phase to form the fermentation broth at (c) comprises draining the first phase from a bottom of a tank.

Example 92 provides the fermentation process of any one of Examples 62-91, further comprising:

(i) collecting the second phase for use as animal feed, collecting the second phase feedstock for an ethanol fermentation plant, processing the second phase to create a solution having a dextrose equivalent number of at least 95, or a combination thereof after (c).

Example 93 provides the fermentation process of any one of Examples 62-92, wherein the microorganism comprises a fungus, a bacterium, or a mixture thereof.

Example 94 provides the fermentation process of Example 93, wherein the fungus comprises a yeast.

Example 95 provides the fermentation process of Example 94, wherein the yeast comprises Saccharomyces cerevisiae, Kluyveromyces lactis, Kluyveromyces marxianus, Yarrowia lipolytica, Pichia kudriavzevii, Schizosaccharomyces pombe, or a mixture thereof.

Example 96 provides the fermentation process of any one of Examples 93-95, wherein the bacteria comprises Streptococcus, Lactobacillus, Bacillus, Escherichia, Salmonella, Neisseria, Acetobactor, Arthrobacter, Aspergillus, Bifdobacterium, Corynebacterium, Pseudomanas, or a mixture thereof.

Example 97 provides the fermentation process of any one of Examples 62-96, wherein the microorganism is an engineered microorganism.

Example 98 provides the fermentation process of Example 97, wherein the engineered microorganism is a yeast that is PDC negative and is not capable of producing ethanol.

Example 99 provides the fermentation process of any one of Examples 62-98, wherein the fermentation wherein the fermentation broth is fermented at (e) to produce the fermentation product and wherein a concentration of the glucose in the fermentation broth is 15 g/L or less and a glucoamylase is added at (f).

Example 100 provides the fermentation process of any one of Examples 62-99, wherein the fermentation broth is fermented at (e) to produce the fermentation product and wherein a concentration of the glucose in the fermentation broth is 15 g/L or less and a glucoamylase is added at (f).

Example 101 provides the fermentation process of any one of Examples 62-100, wherein the fermentation broth is fermented at (e) to produce the fermentation product and wherein a concentration of the glucose in the fermentation broth comprising the first portion of the first phase is in a range of from about 0.1 g/L to about 30 g/L and a glucoamylase is added at (f).

Example 102 provides the fermentation process of any one of Examples 62-101, wherein the fermentation broth comprising the first portion of the first phase is fermented at (e) to produce the fermentation product and wherein a concentration of the glucose is in a range of from about 3 g/L to about 30 g/L and a glucoamylase is added at (f).

Example 103 provides the fermentation process of any one of Examples 63-102, wherein the second portion of the first phase is added at (g) to the fermentation broth to maintain a concentration of the glucose in a range of from about 8 g/L to about 12 g/L.

Example 104 provides the fermentation process of any one of Examples 62-103, wherein the fermentation product comprises less than about 3 g/L of ethanol.

Example 105 provides the fermentation process of any one of Examples 62-104, wherein the fermentation product is substantially free of ethanol.

Example 106 provides the fermentation process of any one of Examples 62-105, wherein the fermentation is stopped after (g) when the concentration of the glucose in the fermentation broth is 0.5 g/L or less.

Example 107 provides the fermentation process of any one of Examples 62-106, wherein the fermentation is stopped after (g) when the concentration of the glucose in the fermentation broth is 0.3 g/L or less.

Example 108 provides the fermentation process of any one of Examples 62-107, wherein the fermentation is stopped after (g) when the concentration of the glucose in the fermentation broth is in a range of from about 0 g/L to about 0.5 g/L.

Example 109 provides the fermentation process of any one of Examples 62-108, wherein the fermentation is stopped after (g) when the concentration of the glucose in the fermentation broth is in a range of from about 0.2 g/L to about 0.4 g/L.

Example 110 provides the fermentation process of any one of Examples 62-109, wherein the method produces less than about 0.5 g/L of isomaltose in the first phase.

Example 111 provides the fermentation process of any one of Examples 62-110, wherein the method produces less than 0.05 g/L isomaltose in the first phase.

Example 112 provides the fermentation process of any one of Examples 62-111, wherein the fermentation product comprises an organic acid or an amino acid, a derivative thereof, or a salt thereof.

Example 113 provides the fermentation process of any one of Examples 62-112, wherein fermentation product the organic acid or amino acid comprises lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof.

Example 114 provides the fermentation process of any one of Examples 62-113, wherein the method produces at least 90 g/L of fermentation product in the fermentation broth at (e), (g), or both.

Example 115 provides the fermentation process of any one of Examples 62-114, wherein the method produces at least 120 g/L of fermentation product in the fermentation broth at (e), (g), or both.

Example 116 provides the fermentation process of any one of Examples 62-115, wherein the method produces about 100 g/L to about 130 g/L of fermentation product in the fermentation broth at (e), (g), or both.

Example 117 provides the fermentation process of any one of Examples 62-116, wherein the method produces about 90 g/L to about 150 g/L of fermentation product in the fermentation broth at (e), (g), or both.

Example 118 provides the fermentation process of any one of Examples 63-117, wherein glucose concentration is monitored by a real-time monitoring system, wherein the real-time monitoring system interfaces with equipment that controls the introduction of the first portion of the first phase, the second portion of the first phase, or both.

Example 119 provides the fermentation process of any one of Examples 62-118, wherein the method is free of adding a trans-glucosidase to the first phase, fermentation broth, or both.

Example 120 provides the fermentation process of any one of Examples 62-119, further comprising:

(j) filtering the microorganism from the fermentation broth and recovering the fermentation product.

Example 121 provides the fermentation process of any one of Examples 62-120, wherein step (f) is performed when the concentration of glucose in the fermentation broth is 40 g/L or less.

Example 122 provides the fermentation process of any one of Examples 62-121, wherein steps (a)-(g) are performed in sequential order.

Example 123 provides a fermentation process, the process comprising:

(a) contacting a starch hydrolysate with an alpha-amylase before contacting the starch hydrolysate with glucoamylase, wherein the starch hydrolysate has a dextrose equivalent number in a range of from about 8 to about 13;

(b) contacting the starch hydrolysate with a glucoamylase with agitation;

(c) ceasing agitation for a sufficient amount of time to allow for settling to form a multi-phase solution, a first phase of the multi-phase solution comprising a saccharide component comprising about 30 wt % to about 80 wt % (for example 30 wt % to 70 wt %, 30 wt % to 60 wt %) based on a total carbohydrate present in the first phase and a second phase of the multi-phase solution comprising a higher total suspended solids amount than the first phase, wherein settling is performed for a time in a range of from about 5 hours to about 13 hours;

(d) draining the first phase to isolate the first phase from the second phase to form a fermentation broth comprising a first portion of the first phase;

(e) heating at least the first phase to a temperature of at least 70° C.;

(f) fermenting the fermentation broth with a PDC negative engineered yeast to produce at least 90 g/L of a fermentation product comprising lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, derivatives thereof, salts thereof, or mixtures thereof until a concentration of glucose is 40 g/L or less; and

(g) contacting the first phase with glucoamylase.

Example 124 provides a fermentation product formed according to the method of any one of Examples 1-123.

1. A fermentation process, the process comprising:
(a) contacting a starch hydrolysate with a glucoamylase with agitation;
(b) ceasing the agitation to allow for settling to form a multi-phase solution, a first phase of the multi-phase solution comprising a saccharide component comprising about 30 wt % to about 70 wt % glucose based on a total carbohydrate present in the first and a second phase of the multi-phase solution comprising a higher amount of total suspended solids than the first phase;
(c) draining the first phase to isolate the first phase from the second phase;
(d) heating at least the first phase to a temperature of at least 70° C.;
(e) adding a first portion of the heated first phase from step (d) to form a fermentation broth and fermenting the fermentation broth with a microorganism to produce a fermentation product in the fermentation broth until a concentration of glucose in the fermentation broth is 40 g/L or less;
(f) contacting the fermentation broth with glucoamylase; and
(g) adding a second portion of the heated first phase to the fermentation broth to maintain a concentration of glucose in a range of from about 1 g/L to about 20 g/L in the fermentation broth.

2. The fermentation process of claim 1, further comprising:
(h) contacting a starch slurry with an alpha-amylase to form a starch hydrolysate that is contacted with the glucoamylase according to (a).

3. The fermentation process of claim 1, wherein the starch hydrolysate introduced at step (a) has a dextrose equivalent number in a range of from about 1 to about 15.

4. (canceled)

5. The fermentation process of claim 1 wherein step (a) further comprises heating the starch hydrolysate to a temperature of at least 80° C.

6. (canceled)

7. The fermentation process claim 1, wherein step (a) further comprises heating the starch hydrolysate to a temperature in a range of from about 80° C. to about 130° C.

8. (canceled)

9. The fermentation process of claim 1, wherein the agitation at (a) is performed for a time in a range of from about 3 hours to about 10 hours.

10. (canceled)

11. The fermentation process claim 1, wherein the settling at (b) is performed for a time in a range of from about 5 hours to about 13 hours.

12. (canceled)

13. (canceled)

14. The fermentation process of claim 1, wherein the second phase comprises proteins, lipids, or a mixture thereof.

15. (canceled)

16. The fermentation process of claim 1, wherein a total amount of the suspended solids in the second phase is at least 1000 ppm.

17.-19. (canceled)

20. The fermentation process of claim 1, wherein a total suspended solids amount of the first phase is less than a total suspended solids amount of the second phase.

21. The fermentation process claim 1, wherein draining is ceased at (c) when a total amount of the suspended solids measured in a pipe downstream of a tank including the multi-phase solution is at least 1000 ppm.

22.-24. (canceled)

25. The fermentation process of claim 1, wherein draining is ceased at (c) at a point to where the first phase comprises a total suspended solids amount of less than 3000 ppm.

26. (canceled)

27. (canceled)

28. The fermentation process of claim 1, wherein step (d) comprises heating to a temperature in a range of from about 80° C. to about 130° C.

29. The fermentation process of claim 1, wherein the draining of the first phase to isolate the first phase from the second phase to form the fermentation broth at (c) comprises draining the first phase from a bottom of a tank.

30. The fermentation process of claim 1, further comprising:
(i) collecting the second phase for use as animal feed, collecting the second phase feedstock for an ethanol fermentation plant, processing the second phase to create a solution having a dextrose equivalent number of at least 95, or a combination thereof after (c).

31. The fermentation process of claim 1, wherein the microorganism comprises a fungus, a bacterium, or a mixture thereof.

32. The fermentation process of claim 31, wherein the fungus comprises a yeast.

33. (canceled)

34. (canceled)

35. The fermentation process of claim 1, wherein the microorganism is an engineered yeast that is PDC negative.

36.-41. (canceled)

42. The fermentation process of claim 1, wherein the fermentation product comprises less than about 3 g/L of ethanol.

43.-50. (canceled)

51. The fermentation process of claim 1, wherein fermentation product is an organic acid or amino acid comprising lactic acid, citric acid, malonic acid, hydroxy butyric acid, adipic acid, lysine, keto-glutaric acid, glutaric acid, 3-hydroxy-proprionic acid, succinic acid, malic acid, fumaric acid, itaconic acid, muconic acid, methacrylic acid, and acetic acid, and derivatives thereof, salts thereof, or mixtures thereof.

52.-127. (canceled)
